SEQUENCE LISTING (21 sequences)

SEQ ID NO: 1 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 2 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 693 bp, synthetic construct
  source 1..693: mol_type = other DNA
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccc

SEQ ID NO: 4 - protein, 231 aa, synthetic construct
MAQSKHGLTKEMTMKYRMEGCVDGHKFVITGEGIGYPFKGKQAINLCVVEGGPLPFAEDILSAAFMYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 5 - DNA, 513 bp, synthetic construct
  source 1..513: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 6 - protein, 170 aa, synthetic construct
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 7 - DNA, 54 bp, synthetic construct
  source 1..54: mol_type = other DNA
gagggcagaggaagtctgctaacatgcggtgacgtcgaggagaatcctggccca

SEQ ID NO: 8 - protein, 18 aa, synthetic construct
EGRGSLLTCGDVEENPGP

SEQ ID NO: 9 - DNA, 57 bp, synthetic construct
  source 1..57: mol_type = other DNA
gctactaacttcagcctgctgaagcaggctggagacgtggaggagaaccctggacct

SEQ ID NO: 10 - protein, 19 aa, synthetic construct
ATNFSLLKQAGDVEENPGP

SEQ ID NO: 11 - DNA, 230 bp, synthetic construct
  source 1..230: mol_type = other DNA
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcag

SEQ ID NO: 12 - DNA, 46 bp, synthetic construct
  source 1..46: mol_type = other DNA
gcctcactgattaagcattggtaataagatacattgatgagtttgg

SEQ ID NO: 13 - DNA, 45 bp, synthetic construct
  source 1..45: mol_type = other DNA
gtgcgaacgcattctggcgtaactgatcataatcagccataccac

SEQ ID NO: 14 - DNA, 27 bp, synthetic construct
  source 1..27: mol_type = other DNA
cgcggaacccctatttgtttatttttc

SEQ ID NO: 15 - DNA, 46 bp, synthetic construct
  source 1..46: mol_type = other DNA
ccaaactcatcaatgtatcttattaccaatgcttaatcagtgaggc

SEQ ID NO: 16 - DNA, 45 bp, synthetic construct
  source 1..45: mol_type = other DNA
gtggtatggctgattatgatcagttacgccagaatgcgttcgcac

SEQ ID NO: 17 - DNA, 67 bp, synthetic construct
  source 1..67: mol_type = other DNA
ctgctgaagcaggctggagacgtggaggagaaccctggacctgtcttcacactcgaagatttcgttg

SEQ ID NO: 18 - DNA, 58 bp, synthetic construct
  source 1..58: mol_type = other DNA
cgtctccagcctgcttcagcaggctgaagttagtagcgggcaaggcggagccggaggc

SEQ ID NO: 19 - DNA, 75 bp, synthetic construct
  source 1..75: mol_type = other DNA
gagggcagaggaagtctgctaacatgcggtgacgtcgaggagaatcctggcccaatggcccagtccaagcacggc

SEQ ID NO: 20 - DNA, 77 bp, synthetic construct
  source 1..77: mol_type = other DNA
ttaagggccggccgcatcagcttgatatcgaattcctgcagatctgctagcgcggaacccctatttgtttatttttc

SEQ ID NO: 21 - DNA, 78 bp, synthetic construct
  source 1..78: mol_type = other DNA
gagttcgtgaccgccgccgggatcactctcggcatggacgagctgtacaaggagggcagaggaagtctgctaacatgc

(19) United States
(12) Patent Application Publication
(10) Pub. No.: US 2025/0361526 A1
(43) Pub. Date: Nov. 27, 2025

(54) ENGINEERED MURINE CYTOMEGALOVIRUS-DERIVED TOOL VIRUS, METHOD OF PRODUCING THE SAME AND APPLICATIONS THEREOF

(71) Applicant: WUHAN INSTITUTE OF VIROLOGY, CHINESE ACADEMY OF SCIENCES, Wuhan (CN)

(72) Inventors: Minhua LUO, Wuhan (CN); Bo YANG, Wuhan (CN); Yuepeng ZHOU, Wuhan (CN); Yongxuan YAO, Wuhan (CN); Mengjie MEI, Wuhan (CN); Jinpeng WU, Wuhan (CN); Wenbo ZENG, Wuhan (CN)

(21) Appl. No.: 18/875,093

(22) PCT Filed: Dec. 14, 2022
(86) PCT No.: PCT/CN2022/138850

§ 371 Date: Dec. 13, 2024

(51) Int. Cl. C12N 15/86; A61K 49/00; C12N 7/00

(52) U.S. Cl. CPC C12N 15/86; C12N 7/00; A61K 49/0004; C12N 2710/16121; C12N 2710/16131; C12N 2710/16143

(57) ABSTRACT

A tool virus derived from genetically engineered MCMV K181 strain for tracing MCMV infection, wherein the tool virus is a recombinant MCMV K181 strain comprising a tracing elements expression cassette and a BAC backbone, wherein the BAC backbone and the tracing elements expression cassette are inserted together between M06 open reading frame and M07 open reading frame of K181 strain genome; wherein the tracing elements expression cassette comprises three tracing elements coding sequences; and wherein the tracing elements expression cassette from 5′ to 3′ sequentially comprises a first fluorescent protein, a first linker, a second fluorescent protein, a second linker, a luciferase and a polyA.

### FIELD OF THE INVENTION

The present invention generally relates to the field of biotechnology, and more particularly to engineered murine cytomegalovirus-derived tool virus, methods for producing the engineered murine cytomegalovirus-derived tool virus, and applications thereof.

### BACKGROUND OF THE INVENTION

Cytomegaloviruses (CMVs) belong to the Betaherpesvirinae subfamily of the Herpesviridae. The global susceptibility of Human cytomegalovirus (HCMV) is 60-100% (Manicklal 2013); the adult infection rate in China is 93.7% (Wen 2018). After hematopoietic stem cell/bone marrow transplantation, active infection rate of HCMV is up 70% (Han 2007), and the mortality rate up to 30% (Arizaheredia 2014; Focosi 2009). HCMV can spread vertically to fetuses, leading to congenital infection. The congenital infection rate is about 0.6-0.7% in the developed countries in Europe and the United States (Swanson 2013), and about 1-5% in developing countries (Manicklal 2013). Hearing loss caused by congenital infection of HCMV accounts for 15% of the total hearing loss in developed countries, but up to 33% in developing countries (Dollard 2007). Congenital infection of HCMV gravely affects the quality of the birth population, bringing staggering mental and economical burdens to families and society. No effective vaccine against HCMV is available; anti-viral drugs for HCMV replication are prone to drug-resistance; the newly marketed Letermovir has drug-resistance, and the cytotoxicity of the drugs has serious side effects on neonates and transplantation recipients of organs and cells. Therefore, it is of great scientific significance and clinical value for clinical intervention to find new intervention targets against HCMV infection. In addition, no vaccine is available to interfere with HCMV infection and related diseases; vaccine evaluation system is indispensable for vaccine research and development.

Studies of influences of congenital infection of CMV on neuronal functions of fetal brain development and hearing, drug evaluation and vaccine evaluation cannot be carried out in cell infection models; moreover, the strict species specificity of CMV infection led to the inability of HCMV infect mice.

As a model for CMV research, murine cytomegalovirus (MCMV) infects mice, and is widely used in the study of cytomegalovirus (CMV) in vivo. In order to trace viral infection, MCMV has been genetically modified to insert into its genome the luciferase gene or EGFP gene for labeling of infected cells, in vivo imaging, tracing the progress of virus infection, realizing the visualization of the progress of virus infection. The modified MCMV is conveniently used for screening drugs, in vivo evaluation of vaccine or neutralizing antibodies, optimization of drug composition and therapy protocols, etc.

Redwood et al. disclose the construction of a bacterial artificial chromosome (BAC) clone of MCMV-K181 Perth strain as a vaccine vector (Redwood 2005). When the BAC cassette containing a GFP-encoding sequence is present during infection, the GFP is expressed inside the infected cells. Their data show that the ORFs m07 to m12 are not essential for the replication of MCMV-K181 strain in vitro (page 3002, right col., lines 4-6), but removal of these ORFs and retention of the BAC cassette attenuated the in vivo growth of deletion mutant virus vARK14 (page 3005, right col., lines 11-13). Furthermore, the vaccine vector is not intended for tracing MCMV infections and visualization.

Farrel et al. disclose MCMV-LUC for live imaging, where an HCMV IE1 promoter-driven luciferase expression cassette was inserted into m157 of a bacterial artificial chromosome (BAC)-cloned MCMV Smith strain genome that was repaired for MCK2 expression (Farrel 2015). Farrel et al. also disclose MCMV-GR for fluorescent labeling of infected cells, where an HCMV IE1 promoter-driven expression cassette was inserted into M157 of MCMV K181 strain by homologous recombination, the expression cassette encoding a floxed enhanced green fluorescent protein (EGFP) coding sequence upstream of nunclear-targeted tdTomoto (Farrel 2015). It is apparent that the results of Farrel et al. indicate that to ensure their expressions, the luciferase and EGFP genes are individually inserted into the genome of MCMV, and each directly driven by HCMV IE1 promoter.

Farrel et al. disclose use of porcine teschovirus-1 2A peptide to generate a luciferase-tagged MCMV for live imaging, where the luciferase gene was inserted downstream of the early lytic gene MCMV M78 (designated M78-LUC) (Farrel 2016). It is apparent that the expression of luciferase is controlled by the expression of M78 gene, i.e., the intensity of luciferase is limited by the expression level of M78 gene.

The inventors of the present invention found in practice that although the existing MCMV-EGFP has a green fluorescence tag, its green fluorescence is weak, which cannot meet the requirements of in time tracing the progress of virus infection in vivo, and the loss of the report gene has been detected in the second generation during propagation. In addition, the expression level and/or fluorescence intensity of fluorescein in MCMV-LUC also limit their application in real-time imaging.

In order to monitor the temporal and spatial distribution of MCMV in mice during MCMV infection and better evaluate the in vivo effects of neutralizing antibodies, vaccines and other potential drugs of MCMV, it is necessary to develop a better murine cytomegalovirus-derived tool virus for tracing virus infection.

### SUMMARY OF THE INVENTION

The present invention provides a genetically engineered MCMV-K181 strain-derived tool virus for tracing virus infection.

In certain embodiments, the tool virus is a recombinant MCMV-K181 strain comprising a tracing elements expression cassette and a BAC backbone, wherein the BAC backbone and the tracing elements expression cassette are inserted together between M06 open reading frame and M07 open reading frame of MCMV-K181 strain genome; wherein the tracing elements expression cassette comprises three tracing elements coding sequences; and wherein the tracing elements expression cassette from 5 ‘to 3’ sequentially comprises a first fluorescent protein, a first linker, a second fluorescent protein, a second linker, a luciferase and a polyA, and is transcribed into a transcript, resulting in three tracing elements after translation, namely, the first fluorescent protein, the second fluorescent protein and the luciferase.

In certain embodiments of the tracing system, the BAC backbone comprises a DNA replication origin ori, a repE gene, a sopA gene, a sopB gene, a sopC gene, an ori2 replicon, a prokaryotic transcription terminator, and an ampicillin resistance gene.

In certain embodiments of the tracing system, the insertion site of the BAC backbone and tracing elements expression cassette between the M06 open reading frame and M07 open reading frame of MCMV-K181 strain genome is between 6522 and 6545.

In certain embodiments of the tracing system, the first and second fluorescent proteins are selected from the group consisting of GFP (green fluorescent protein), eGFP (enhanced green fluorescent protein), mGFP (membrane bound form of eGFP), sfGFP (super folder green fluorescent protein), mNeonGreen, StayGold, EYFP (enhanced yellow fluorescent protein), ECFP (enhanced cyan fluorescent protein) EBFP2 (enhanced blue fluorescent protein 2), tdTomato, MRFP (monomer red fluorescent protein), mRb3, mScarlt, DsRed, mCherry, Ypet, mKO, mkate and iRFP.

In certain embodiments of the tracing system, the first fluorescent protein is eGFP with an amino acid sequence represented by SEQ ID NO. 2, and the second fluorescent protein is ZsGreen1 with an amino acid sequence represented by SEQ ID NO. 4.

In certain embodiments of the tracing system, the luciferase is NanoLuc with an amino acid sequence represented by SEQ ID NO. 6.

In certain embodiments of the tracing system, the first linker and the second linker encode a linker polypeptide, wherein the linker polypeptide comprises at least adjacent glycine and proline.

In certain embodiments of the tracing system, the linker polypeptide is T2A with an amino acid sequence represented by SEQ ID NO. 8, or P2A with an amino acid sequence represented by SEQ ID NO. 10.

In certain embodiments of the tracing system, the polyA is SV40 pA with a nucleotide sequence represented by SEQ ID NO. 11.

In certain embodiments of the tracing system, the tracer factor expression cassette further comprises a promoter, which is selected from the group consisting of CMV promoter, EF1a promoter, SV40 promoter, PGK1 promoter, Ubc promoter, human beta actin promoter, CAG promoter, and HIF alpha promoter.

The objectives and advantages of the invention will become apparent from the following detailed description of preferred embodiments thereof in connection with the accompanying drawings.

### BRIEF DESCRIPTION OF THE DRAWINGS

Preferred embodiments according to the present invention will now be described with reference to the Figures, in which like reference numerals denote like elements.

FIG. 1 is a schematic diagram showing the structure of MCMV-K181-eGFP (K181-eGFP) (Redwood 2005).

FIG. 2 is a schematic diagram showing the structure of MCMV-K181-V1 (K181-V1) without SV40 PolyA element in the tracing elements expression cassette.

FIG. 3 is a schematic diagram showing the structure of MCMV-K181-eGFP-NLuc-ZsGreen1 (K181-Nluc/Green) with SV40 PolyA element in the tracing elements expression cassette.

FIG. 4 is an electrophoretic image showing the PCR results.

FIG. 5 shows the fluorescence intensity of NIH3T3 cells infected by K181-eGFP, K181-V1 and K181-Nluc/Green recombinant viruses.

FIG. 6 shows the Western blot results of protein expressions in NIH3T3 cells infected with K181-eGFP and K181-Nluc/Green recombinant viruses.

FIG. 7 shows the growth curves of K181-eGFP and K181-Nluc/Green recombinant viruses in NIH3T3 cells.

FIG. 8 shows the green fluorescence signals of NIH3T3 cells after being infected with the K181-eGFP and K181-Nluc/Green recombinant viruses with a MOI of 0.01 for 24 hours.

FIG. 9 shows the activity of NanoLuc gene of K181-Nluc/Green recombinant virus in NIH3T3 cells.

FIG. 10 is a curve graph showing the relationship between the virus titer in the supernatant of NIH3T3 cells infected with K181-Nluc/Green recombinant virus and the activity of the NanoLuc gene.

FIG. 11 is flow chart showing that ICR mice were infected with K181-Nluc/Green recombinant virus.

FIG. 12 shows the live imaging of the control group (PBS) and the ganciclovir (GCV) treatment group of ICR mice infected with K181-Nluc/Green recombinant virus (one representative result was displayed in each group).

FIG. 13 shows the quantitative analysis results of fluorescence values of PBS and GCV groups.

FIGS. 14 and 15 show the GFP and Nluc signals of each organ in ICR mice placebo group (DMSO) and ganciclovir treatment group (GCV) 7 days after infection with K181-Nluc/Green recombinant virus.

### DETAILED DESCRIPTION OF THE INVENTION

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, for example, Molecular Cloning: A Laboratory Mannual, third edition (Sambrook and Russel, 2001); Current Protocols in Molecular Biology (F. M. Ausubel et al., eds., 1987, updated to 2001).

Murine cytomegalovirus (MCMV)-K181 strain (GenBank: AM886412.1) has a genome about 230 kb, encoding genes from m01 to m170, a total of over 170 open reading frames (ORFs).

In order to develop MCMV-K181 strain-derived tool virus for tracing virus infection, the inventors of the present invention conducted detailed and extensive studies, including the choice of tracing elements, the insertion points of tracing elements, and the constructions of expression cassettes of tracing elements.

The present invention provides an engineered MCMV-K181 strain-derived tool virus for tracing virus infection; the tool virus is a recombinant MCMV-K181 straom, including a tracing elements expression cassette, inserted into K181-BAC backbone; BAC and the tracing elements expression cassette together inserts into MCMV-K181 strain genome between the ORFs of M06 and M07. The tracing elements expression cassette includes encoding sequences of three tracing elements; sequentially from 5′ to 3′, first fluorescent protein, first linker, second fluorescent protein, second linker, luciferase and PolyA are being translated into one transcript; after translation, three individual tracing elements are produced, i.e. first fluorescent protein, second fluorescent and luciferase. The tool virus effectively enhances the tracing fluorescence intensity and sensitivity; using live imaging can directly non-invasive observe the initiation of MCMV infection and the viral replication levels of the locations/organs to which the infection spreads; thereby the in time distributions and infection levels of MCMV in the body of mice can be evaluated more accurately and broadly.

In certain embodiments, the BAC backbone includes DNA replication origin ori, repE gene, sopA gene, sopB gene, sopC gene, ori2 replicon, prokaryotic transcription terminator and ampicillin resistance gene. In certain embodiments, the insertion site of the BAC backbone and tracing elements expression cassette in MCMV-K181 strain genome is located between M06 and M07 open reading frames, as long as the functions of M06 and M07 open reading frames are not affected. In certain embodiments, the insertion site of the BAC skeleton and tracing elements expression cassette in MCMV-K181 strain genome is located between 6522 and 6545.

In certain embodiments, the first and second fluorescent proteins can be any fluorescent protein available at present and in the future. Fluorescent protein can be wild type or its variant, as long as its fluorescence intensity is not attenuated. Suitable fluorescent proteins include GFP (green fluorescent protein), eGFP (enhanced green fluorescent protein), mGFP (membrane bound form of eGFP), sfGFP (super folder green fluorescent protein), mNeonGreen, StayGold, EYFP (enhanced yellow fluorescent protein), ECFP (enhanced cyan fluorescent protein), EBFP2 (enhanced blue fluorescent protein 2), tdTomato, MRFP (monolayer red fluorescent protein), mRb3, MScarlt, DsRed, mCherry, Ypet, mKO, mkate, iRFP, etc. In certain embodiments, the first and second fluorescent proteins are the same fluorescent protein. In certain embodiments, the first and second fluorescent proteins are different fluorescent proteins. In certain embodiments, the first fluorescent protein is eGFP, its coding nucleotide sequence is represented by SEQ ID NO. 1, and its amino acid sequence by SEQ ID NO. 2; the second fluorescent protein is ZsGreen1, its coding nucleotide sequence is represented by SEQ ID NO. 3, and its amino acid sequence by SEQ ID NO. 4.

Luciferase is a generic term for enzymes that can produce bioluminescence in nature. The bioluminescence based on luciferase does not require excitation, has strong specificity, is less absorbed by tissues, and has no self-luminescence in animals, possessing the advantages of low background and high signal-to-noise ratio. Through in vivo imaging system, non-invasive methods can be used to observe the luminescence in live animals. By comparing Firefly luciferase, Renilla Luciferase and NanoLuc luciferase, the inventors of the present invention found that after inserting into the genome of MCMV-K181 strain, NanoLuc luciferase does not affect in vitro and in vivo viral replication and spreading, has more excellent sensitivity and is convenient for quantitative detection. In certain embodiments, the luciferase is NanoLuc, its coding nucleotide sequence is represented by SEQ ID NO. 5, and its amino acid sequence by SEQ ID NO. 6.

In certain embodiments, the first linker and the second linker encode a linker polypeptide, where the linker polypeptide contains at least two adjacent amino acids, and the probability of forming peptide bonds between them is very low. Therefore, separate tracing elements are formed after translation. In certain embodiments, the at least two adjacent amino acids are glycine and proline. In certain embodiments, the linker polypeptide is T2A, its coding nucleotide sequence is represented by SEQ ID NO. 7, and its amino acid sequence by SEQ ID NO. 8. In certain embodiments, the linker polypeptide is P2A, its coding nucleotide sequence is represented by SEQ ID NO. 9, and its amino acid sequence by SEQ ID NO. 10. In certain embodiments, their variants can be used; “variant” is defined as a polypeptide that has at least 90%, preferably 95%, more preferably 98%, or even more preferably 99% identity with an amino acid sequence represented by a sequence number, as long as changes in the variant do not affect its function.

In certain embodiments, the PolyA is SV40 pA, and its nucleotide sequence is represented by SEQ ID NO. 11.

In certain embodiments, the tracing elements expression cassette also contains an eukaryotic promoter from a virus or mammal, such as CMV promoter, EF1a promoter, SV40 promoter, PGK1 promoter, Ubc promoter, human beta action promoter, CAG promoter, HIF alpha promoter, etc. In certain embodiments, the promoter contained in the tracing elements expression cassette is CMV promoter.

The following embodiments are provided for the purpose of illustrating the application of the principles of the present invention; they are by no means intended to be the coverage of the present invention.

### Embodiment 1. Cells and Cell Culture

NIH3T3 cell (ATCC #CRL-1658) was obtained from ATCC, and maintained in our lab; no mycoplasma contamination was detected. Cells were cultured in DMEM (Gibco/Life Technologies) supplemented with 10% FBS (Gibco/Life Technologies) and penicillin (100 U/mL)-streptomycin (100 μg/mL); culture medium was changed every 2 days. Cells were cultured at 37° C. with 5% CO₂.

### Embodiment 2. Statistics Analysis

Each experiment was performed in triplicate, and the results are expressed in mean±SD (standard deviation) from at least three independent experiments of cells or animals. Appropriate statistical tests were used for data analysis, including Student's t-test analysis. When p<0.05, the difference was significant.

### Embodiment 3. Template Plasmids

pHAGE-CMV-MCS-IRES-ZsGreen1 plasmid was maintained in our lab; pEGFP-N1 (Cat. #637402, Takara); pNL1.1 plasmid (NanoLuc) vector (Cat. #PAN1001, Promega).

### Embodiment 4. Reagents

PrimeSTAR Max DNA Polymerase (Cat. #R045B, Takara, Japan); Agarose (Bionest Company, Spain); LipoFectMax (Cat. #FP310, ABP Biosciences, USA); Glue recovery kit (Cat. #D2500-03, Omega Bio Tek Company, USA).

### Embodiment 5. Construction and Amplification of Recombinant Virus: MCMV-AmpR-SV40 (PloyA)-NanoLuc-P2A-ZsGreen1-T2A-eGFP (designated as K181-Nluc/Green)

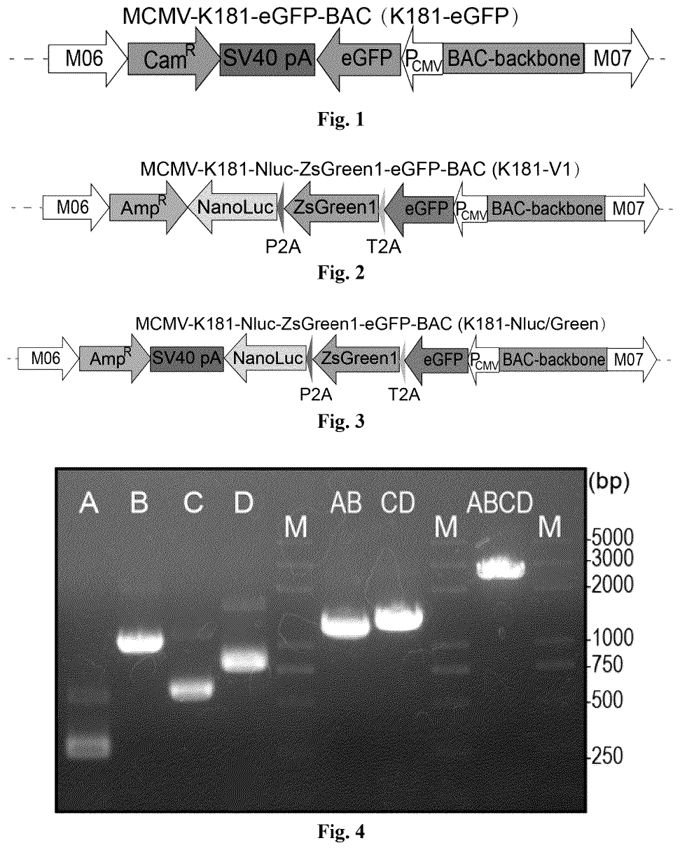

K181-Nluc/Green was derived from MCMV-K181-eGFP (K181-eGFP, FIG. 1) (Redwood 2005). FIG. 2 shows the block schematic diagram of MCMV-K181-V1 (K181-V1) containing a tracing elements expression cassette without SV40 PolyA element. FIG. 3 shows the block schematic diagram of K181-Nluc/Green containing a tracing elements expression cassette with SV40 PolyA element.

### (1) Construction of Expression Cassette

AmpR-SV40 (PolyA)-NanoLuc-P2A-ZsGreen1-T2A expression cassette was constructed by PCR amplification of 4 fragments and then by fusion with PCR. The specific operations were as follows:

Using pEGFP-N1 plasmid as template, F-AmpR-SV40PA (SEQ ID NO. 12) and R-Nluc SVPA (SEQ ID NO. 13) as primers, fragment A (276 bp) was amplified by PCR; using pHAGE-CMV-MCS-IRES-ZsGreen1 plasmid as template and F-AmpR (SEQ ID NO. 14) and R-AmpR-SV40PA (SEQ ID NO. 15) as primers, fragment B (1038 bp) was amplified by PCR; using pNL1.1 plasmid as template, F-Nluc-SV40PA (SEQ ID NO. 16) and R-P2A-Nluc (SEQ ID NO. 17) as primers, fragment C (578 bp) was amplified by PCR; using pHAGE-CMV-MCS-IRES-ZsGreen1 plasmid as template and F-P2A-ZG (SEQ ID NO. 18) and R-T2A-ZG (SEQ ID NO. 19) as primers, fragment D (784 bp) was amplified by PCR.

Using F-AmpR (SEQ ID NO. 14) and R-Nluc-SV40PA (SEQ ID NO. 1213) as primers and the fragments A and B as templates, fragment AB (1218 bp) was obtained by fusion PCR amplification; using F-Nluc-SV40PA (SEQ ID NO. 16) and R-T2A-ZG (SEQ ID NO. 19) as primers, the fragments C and D as templates, fragment CD (1340 bp) was obtained by fusion PCR amplification; then using F-HR (SEQ ID No. 20) and R-HR SEQ ID No. 21 as primers, and the fragments AB and fragment CD as templates, fragment ABCD (2614 bp) was obtained by fusion PCR amplification, i.e., left-arm-AmpR-SV40 (PolyA)-NanoLuc-P2A-ZsGreen1-T2A-right-arm expression cassette with homologous arms.

The total volume of PCR reaction system (PrimeStar MAX DNA Polymerase, TaKaRa Company, Japan) was 20 μl. The PCR reaction system was as follows:

| | | |
|---|---|---|
| PrimeSTAR Max premix | 10 | μL |
| F-upstream primer | 1 | μL |
| R-downstream primer | 1 | μL |
| Template DNA | 1 | μL |
| ddH2O | 7 | μL |

The conditions for PCR amplification were: pre-denaturation at 95° C. for 1 min; denaturation at 98° C. for 15 s, annealing at 60° C. for 15 s, extension at 72° C. for 10 s/kb, 34 cycles in total; extension for 5 min after the last 72° C. After each round of amplification, the PCR product was subjected to 1% agarose (Biotest Company, Spain) gel electrophoresis. The purification steps were completely in accordance with the instructions of the kit (Omega Company, USA). Finally, DNA was eluted with preheated deionized water. The primers used in amplification are shown in Table 1, and the electrophoresis results of PCR amplification are shown in FIG. 4.

TABLE 1
Sequences of PCR amplification primers

| | Primer | |
|---|---|---|
| Fragment | designation | Primer sequence and corresponding SEQ ID NO. |
| A | F-AmpR- | GCCTCACTGATTAAGCATTGGTAATAAGATACATTGATGA |
| | SV40PA | GTTTGG (SEQ ID NO. 12) |
| | R-Nluc- | GTGCGAACGCATTCTGGCGTAACTGATCATAATCAGCCAT |
| | SV40PA | ACCAC (SEQ ID NO. 13) |
| B | F-AmpR | CGCGGAACCCCTATTTGTTTATTTTTC (SEQ ID NO. 14) |
| C | R-AmpR- | CCAAACTCATCAATGTATCTTATTACCAATGCTTAATCAG |
| | SV40PA | TGAGGC (SEQ ID NO. 15) |
| | F-Nluc- | GTGGTATGGCTGATTATGATCAGTTACGCCAGAATGCGTT |
| | SV40PA | CGCAC (SEQ ID NO. 16) |
| | R-P2A-Nluc | CTGCTGAAGCAGGCTGGAGACGTGGAGGAGAACCCTGGA |
| | | CCTGTCTTCACACTCGAAGATTTCGTTG (SEQ ID NO. 17) |
| D | F-P2A-ZG | CGTCTCCAGCCTGCTTCAGCAGGCTGAAGTTAGTAGCGGG |
| | | CAAGGCGGAGCCGGAGGC (SEQ ID NO. 18) |
| | R-T2A-ZG | GAGGGCAGAGGAAGTCTGCTAACATGCGGTGACGTCGAG |
| | | GAGAATCCTGGCCCAATGGCCCAGTCCAAGCACGGC (SEQ |
| | | ID NO. 19) |
| ABCD | F-HR | TTAAGGGCCGGCCGCATCAGCTTGATATCGAATTCCTGCA |
| | | GATCTGCTAGCGCGGAACCCCTATTTGTTTATTTTTC (SEQ |
| | | ID NO. 20) |
| | R-HR | GAGTTCGTGACCGCCGCCGGGATCACTCTCGGCATGGACG |
| | | AGCTGTACAAGGAGGGCAGAGGAAGTCTGCTAACATGC |
| | | (SEQ ID NO.21) |

(2) Activating Escherichia coli Containing K181-eGFP
(a) scribing the E coli DY380 containing K181-eGFP on LB solid plate containing chloramphenicol, and culturing it overnight at 32° C.;
(b) inoculating a single clone into 5 mL of LB medium containing chloramphenicol, and incubating it overnight at 32° C. in a shaker;
(c) transferring 400 μl activated bacterial solution to 40 ml liquid medium, and incubating on 32° C. shaking table for about 3 hours, so that the OD600 value of bacterial solution was about 0.55-0.6;
(d) incubating in 42° C. water bath for 15 minutes to activate temperature-sensitive recombination enzyme;
(e) quickly placing the bacterial solution on ice for 10 minutes;
(f) centrifugating at 4000 rpm, at 4° C. for 10 minutes, and discarding the supernatant;
(g) resuspending the bacteria pellet with sterilized ultrapure water, centrifugating at 4000 rpm at 4° C. for 10 minutes, and discarding the supernatant;
(h) resuspending the bacteria pellet with 10% glycerol, and centrifugating at 4000 rpm at 4° C. for 10 minutes, discarding the supernatant;
(i) repeating step (h) once;
(j) resuspending the bacteria pellet with 80 μl ultrapure water containing 10% glycerol.
   (3) Obtaining K181-Nluc/Green by electroporation of AmpR-SV40 (PolyA)-NanoLuc-P2A-ZsGreen1-T2A expression cassette and homologous recombination.

300 ng expression cassette DNA (about 10 μl) was added into the activated Escherichia coli containing K181-eGFP genome prepared in step (2), mixing well; then adding the mixture into an electroporation cup and placing on ice; electric shock condition was 1.6/1.8 kv, 25 μF, 200 Ω, 1 mm; after electric shock, adding the non-resistant LB culture medium into the electroporation cup and quickly transferring the bacterial solution to 1.5 ml EP tube, and incubating in a 32° C. shaker for 1-2 hours; then centrifugating at 2000 rpm for 5 min, discarding the supernatant; resuspending the bacteria in 30 μl sterile water, and evenly smearing on solid LB plate (containing ampicillin resistance), and culturing at 32° C. for 36-48 h; single clones were selected for PCR validation.

### (4) Rescuing K181-Nluc/Green Recombinant Virus

Inoculating the correctly identified single clone bacteria containing K181-Nluc/Green into 200 ml LB medium (containing ampicillin resistance), incubating them on a shaking table at 32° C. overnight, and then extracting K181-Nluc/Green BAC genomic DNA with a plasmid extraction kit (Cat. #740410.50, MN company, Germany), operating according to the instructions, and finally dissolving in 100 μl preheated sterile deionized water.

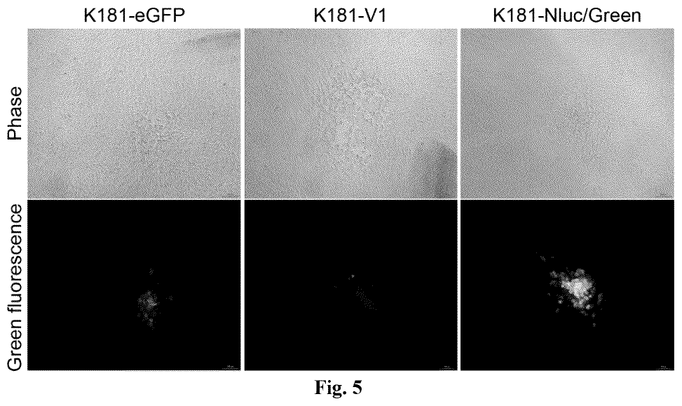

One day before transfection, NIH3T3 cells were passaged to 6-well plate at a density of 4×10⁵ cells/well (Corning Company, USA), reaching 60-80% confluency for transfection. The K181-Nluc/Green-BAC genomic DNA extracted above was transfected into NIH3T3 cells. The transfection mixture was prepared as follows: 3 μg circular K181-Nluc/Green-BAC DNA and DMEM medium without serum and antibiotics were combined into 125 μl Mixture A; at the same time, 5 μl LipoFectMax reagent and DMEM medium without serum and antibiotics were combined into 125 μl Mixture B; after being placed at room temperature for 5 minutes, mixing Mixture A and Mixture B, blowing and mixing with a pipette to obtain Mixture C, and placing at room temperature for 20 minutes. Then removing the cell culture medium, washing the cells with preheated PBS, replacing them with DMEM culture medium without serum and antibiotics, adding transfection Mixture C, and culturing them in a cell incubator at 37° C. 2-3 hours later, sucking out the transfection mixture and adding DMEM complete medium; obvious cytopathic effect and green fluorescence were observed 48 hours after transfection of K181-Nluc/Green infectious single clones, which indicated that our rescue of infectious single clones was successful, as shown in FIG. 5. Then continuing the culture until all the cells were cytopathic. Collecting the cell culture medium to obtain K181-Nluc/Green recombinant virus. After sub-packaging, freezing them at −80° C. Comparing the three recombinant viruses, it was found that the fluorescence of K181-Nluc/Green recombinant virus is significantly stronger than the original K181-eGFP and K181-V1 without SV40 PolyA element (FIG. 5).

### (5) Detection of Protein Expression of Recombinant Viruses

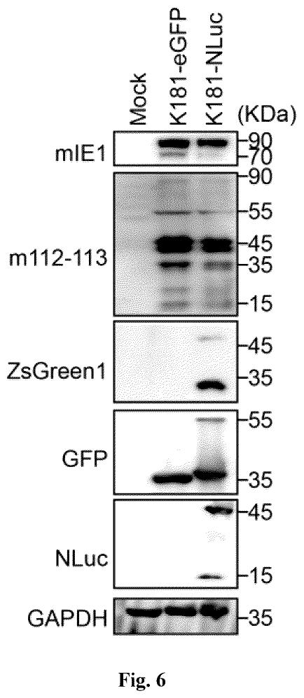

NIH3T3 cells were cultured in 100 mm culture dishes (Corning Company, USA) at 37° C. and 5% CO₂. After the cells were completely adhered to the plates, they were infected with K181-eGFP and K181-Nluc/Green recombinant virus with MOI=1, respectively. After 2 hours of adsorption in a 37° C., 5% CO₂ incubator, replacing the virus inoculum in the culture dish with fresh DMEM complete medium. After 24 hours of infection, the infected cells were scraped with a cell scraper. Cells were collected by centrifugation at 4° C. at 1000 rmp for 5 minutes. Washing the cells with precooled PBS once, continuing centrifugating to remove the supernatant, and gently transferring the cells to the precooled centrifuge tube. Adding 50 μl lysate buffer containing protease inhibitor (Cat. #P0013, Biyuntian Company, China) to the cell precipitation, crushing cells with a sonicator (25% power, running 3 seconds, pausing 3 seconds, a total running of 18 seconds, 3 times of repetition), and then the protein content was determined with the Barford method. Adding 5× loading buffer solution (0.25 mol/l pH6.8 Tris HCl, 0.5 mol/l dithiobetitol, 10% SDS, 0.5% bromophenol blue, 50% glycerol), loading 30 μg total protein for SDS polyacrylamide gel electrophoresis (Bio Red, USA). After the electrophoresis was completed, the membrane transfer was carried out. The polyvinylidene fluoride (PVDF) membrane was first treated with methanol for 2 minutes, then it was cleaned with deionized water once, soaked in the membrane transfer buffer. The membrane transfer condition was constant current of 200 mA in the ice bath for 120 minutes (Bio Red, USA). After membrane transfer, immediately washing the membrane with TBST solution for 3 minutes, and then blocking it with TBST containing 5% skimmed milk for 1 hour. Then, after washing the membrane with TBST containing 1% Tween-20 for three times, the mIE1 mouse monoclonal antibody (gift from Professor Tang Qiyi of Harvard Medical School in the United States), the pM112-113 rabbit polyclonal antibody (produced by Pujian Company, China, for our Laboratory), the GFP rabbit polyclonal antibody (Cat. #50,430-2-AP, Proteintech Company, USA), the ZsGreen1 mouse monoclonal antibody (Cat. #D199,984, China Bioengineering Company), NanoLuc mouse monoclonal antibody (Cat. #N7000, Promega, USA), GAPDH rabbit polyclonal antibody (Cat. #10494-1-AP, Proteintech, USA). After membrane washing, incubating the second antibody and washing the membrane again. Finally, carrying out chemiluminescence development (Alpha Company, USA), and the results are shown in FIG. 6.

### Embodiment 6. Growth Comparison of Recombinant Viruses K181-eGFP and K181-Nluc/Green

NIH3T3 cells were plated into 6-well plate at a density of 5×10⁵ cells/well. After the cells adhered to the plate, K181-eGFP and K181-Nluc/Green respectively infected the cells with MOI of 0.01 (set as 0 hour after infection). Changing the culture medium after incubation for 2 hours. The infected cells were cultured with DMEM complete medium; then the culture supernatants were collected at different time points 2, 4, 6, 8, 10 and 12 days after virus infection and stored at −80° C. After all virus samples were collected, the virus titer of each sample was according to the following steps.

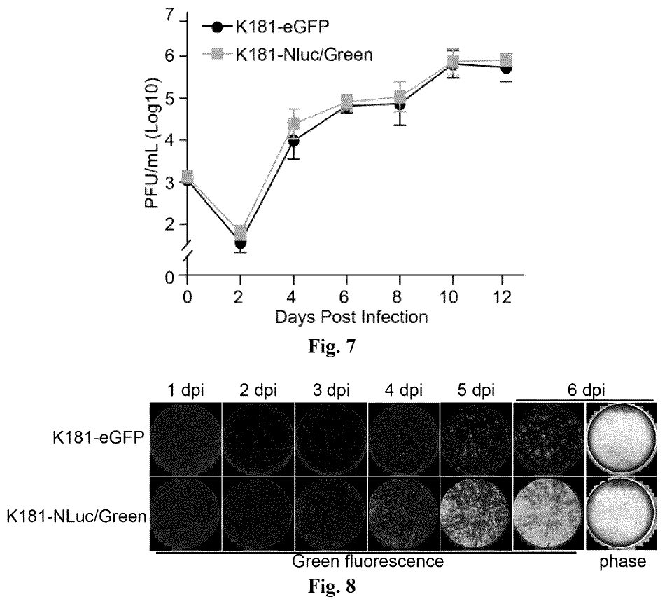

The steps of virus titer determination were as follows: the day before virus determination, NIH3T3 cells were passaged to 24 well plate at a density of 1.5×10⁶ cells/well. On the day of virus titration, the collected culture supernatant samples of K181-eGFP and K181-Nluc/Green infections were diluted in 10-fold sequence with DMEM complete medium, and each concentration was determined three times repeatedly; 200 μl virus solution was added into each well. After 3 hours, quickly mixing 19 ml preheated DMEM complete culture medium with 6 ml microwave-melted 2% sterilized agar, cooling to about 40° C., and adding 1 ml/well into the 24 well plate. After 72 hours of incubation, the number of plaques appearing at the lowest concentration did not increase. Then the plaques were counted to calculate the titer, and the results are shown in FIG. 7.

### Embodiment 7. Fluorescence Intensity of Recombinant Viruses K181-eGFP and K181-Nluc/Green in NIH3T3 Cells

NIH3T3 cells were infected by K181-eGFP and K181-Nluc/Green recombinant viruses at MOI of 0.01. The green fluorescence signal was observed every 24 hours after infection. Images were taken under the same conditions. K181-Nluc/Green shows significantly stronger green fluorescence signal than K181-eGFP (the results are shown in FIG. 8).

### Embodiment 8. Application of Recombinant Virus K181-Nluc/Green in Virus Replication Research

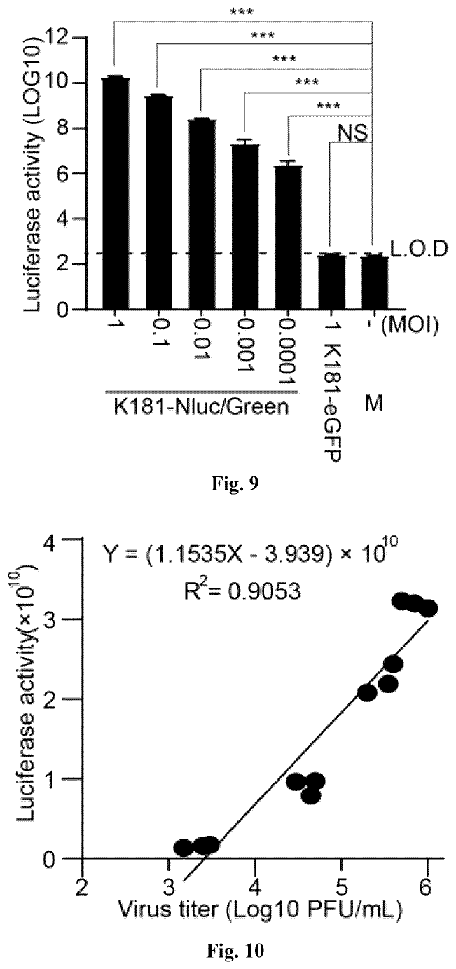

NIH3T3 cells in good growth condition were plated into 12-well plate at a density of 1.5×10⁶ cells/well, and the cells were cultured at 37° C. and 5% CO₂. After the cells adhered to the wells, the cells were infected with recombinant viruses K181-eGFP virus and K181-Nluc/Green respectively with different MOI. After 48 hours, collecting cells and culture supernatant samples, and titrating the viruses in the supernatant of cell culture medium according to the plaque determination method described in Embodiment 6. The activity of NanoLuc was determined according to the instructions of Nano-Glo Luciferase Assay System (Cat. #N1120, Promega, USA). The specific operations were as follows:
a) removing the excess cell culture medium and adding 200 μl Glo Lysis buffer, lysing cells on a shaking table at room temperature for 15 minutes;
b) taking 10 μl lysis solution for each well to add into 50μ Nano-Glo Luciferase Assay Reagent balanced at room temperature, and mixing evenly; the activity of the NanoLuc reporter gene was detected using luminometer; the results are shown in FIG. 9. The relationship between the virus titer in the cell supernatant and the activity of the NanoLuc reporter gene is shown in FIG. 10.

The results showed that after infection of recombinant virus K181-Nluc/Green for 48 hours, the activity of NanoLuc is positively correlated with infection MOI; the virus titers of the culture supernatants are expressed as the logarithm values at the base of 10; the numerical values have a positively correlated linear relationship with the activity of NanoLuc.

### Embodiment 9. Evaluation of the Anti-Viral Protective Effect of Ganciclovir (GCV) on the Transmission of MCMV in Newborn Rats Using Recombinant Virus K181-Nluc/Green

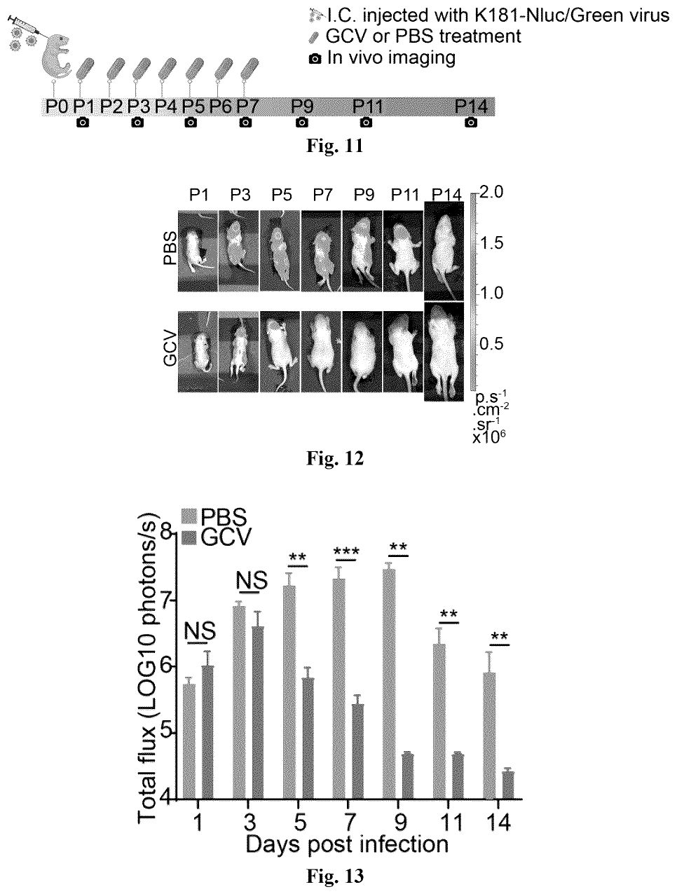

To verify whether recombinant virus K181-Nluc/Green can be applied to evaluate potential drugs, neutralizing antibodies, vaccines, etc. against CMV in vivo. The inventors of the present invention carried out in vivo challenge test with ICR mice in the Animal Biosafety Laboratory (ABSL-2). FIG. 11 shows the detailed flow chart. Twelve newborn ICR mice were divided into three groups: control group (PBS), placebo group (DMSO) and ganciclovir (GCV) treatment group. By intracerebroventricular injection (I.C.), 1 μl (4×10⁵ PFU) K181-Nluc/Green virus solution was injected into the lateral ventricles of newborn mice (P0) to establish the brain infection model of MCMV virus. After 1 day of challenge, 50 mg/kg GCV was given to mice every day until the 7th day after infection, and the Nanoluc substrate (Cat. #N1120, Promega, USA) was intraperitoneally injected on the 1st, 3rd, 5th, 7th, 9th, 11th and 14th days after infection. The IVIS small animal imaging system (Caliper Life Sciences, USA) was used to in time monitor the MCMV infection, progress of infection and virus distribution in vivo in mice (FIG. 12, one representative result was displayed for each group), and the fluorescence value was quantitatively scanned and analyzed by the imaging software (Living Image software, version 4.5; Caliper Life Sciences, USA); the temporal and spatial distributions of virus infection were obtained; the results are shown in FIG. 13.

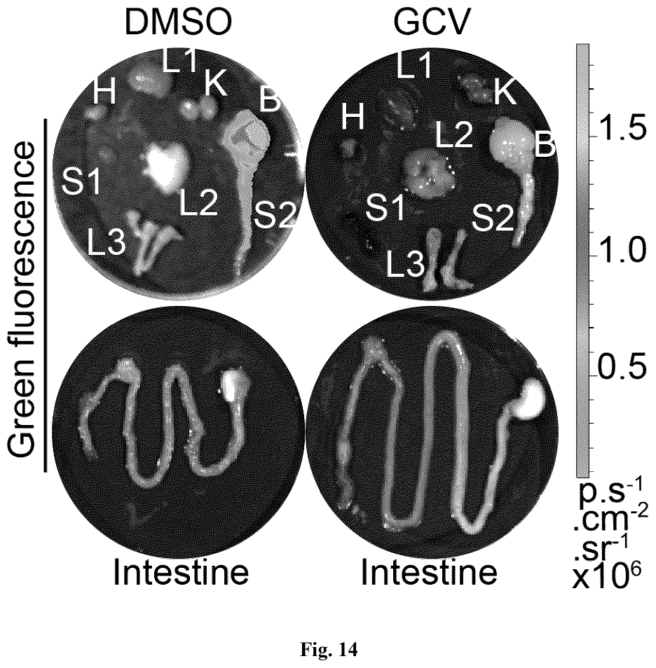

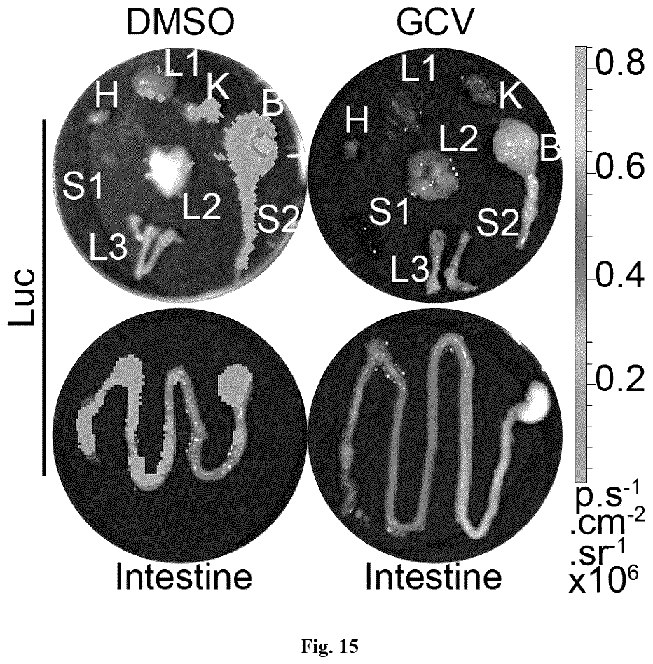

In addition, seven days after the mice were infected, one mouse was randomly selected from the placebo (DMSO) group and the GCV treatment group, and Nanoluc substrate was injected intraperitoneally. The distribution of MCMV in mice was observed through IVIS small animal imaging system (Caliper Life Sciences, USA), and then the mice were quickly dissected. The organs and tissues of mice were taken, and the content of MCMV virus in organs and tissues of mice in different groups was evaluated by fluorescence activity. The experimental results are shown in FIG. 14 and FIG. 15. The results show that 1) the placebo group after being infected by the virus: detecting Nluc fluorescence signals in brain, liver, spleen, lung, kidney, bone marrow, spine, etc., and significant GFP signals in the spine and brain, indicating that the model can be used to monitor the temporal and spatial distribution of MCMV infection in vivo; 2) GCV-treatment group: after 7 days of GCV treatment, no detection of apparent Nluc or GFP signals in different organs of mice; these results demonstrated that GCV treatment can effectively reduce the MCMV replication and spreading in vivo and (FIGS. 14 and 15). This shows that recombinant virus K181-Nluc/Green can be used to evaluate the in vivo anti-viral effects of anti-viral drugs, and also to evaluate the in vivo effects of neutralizing antibodies, vaccines and other potential drugs against MCMV.

In summary, the present invention has provided a visual tracing system for MCMV, which, in combination with green fluorescent protein, luciferase reporter gene and living imaging system, can non-invasively detect the replication level of MCMV in vivo. At the same time, the present invention has carried out research on ICR newborn mice to verify the efficacy of anti MCMV virus drugs in vivo, illustrating an application scenario for using this model to develop anti MCMV virus drugs.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

The Sequence Listing is with a file name of sequence list.xml, a production date of 2022 Dec. 7, and a size of 23,000 bytes.

### REFERENCES

Arizaheredia, E. J., Nesher, L, and Roy F Chemaly, R. F. (2014) Cytomegalovirus diseases after hematopoietic stem cell transplantation: A mini-review, Cancer Lett, 342:1-8
Dollard, S. C., Grosse, S. D., and Ross, D. S. (2007) New estimates of the prevalence of neurological and sensory sequelae and mortality associated with congenital cytomegalovirus infection, Rev Med Virol, 17:355-63
Farrel, H. E., Davis-Poynter, N., Bruce, K., Lawler, C., Dolken, L., Mach, M., Stevenson, P. G. (2015) Lymph Node Macrophages Restrict Murine Cytomegalovirus Dissemination. J Virol. 89:7147-7158
Farrell, H., Oliveira, M., Macdonald, K., Yunis J., Mach M., Bruce K., Stevenson, P., Cardin, R., and Davis-Poynter, N. (2016) Luciferase-tagged wild-type and tropism-deficient mouse cytomegaloviruses reveal early dynamics of host colonization following peripheral challenge Journal of General Virology. 97:3379-3391
Focosi, D., Sordi, E., Papineschi, F., Benedetti, E., Galimberti, S., and Petrini, M. (2009) Fatal ongoing human cytomegalovirus reactivation during high-dose melphalan and autologous stem cell transplantation. J Med Virol, 81:857-60
Hall, M. P., Unch, J., Binkowski, B. F., Valley, M. P., Butler, B. L., Wood, M. G., Otto, P., Zimmerman, K., Vidugiris, G., Machleidt, T., Robers, M. B., Benink, H. A., Eggers, C. T., Slater, M. R., Meisenheimer, P. L., Klaubert, D. H., Fan, F., Encell, L. P., and Wood, K. V . . . (2012) Engineered luciferase reporter from a deep sea shrimp utilizing a novel imidazopyrazinone substrate. ACS Chem Biol, 7:1848-57
Han, X. Y. (2007) Epidemiologic analysis of reactivated cytomegalovirus antigenemia in patients with cancer, J Clin Microbiol, 45:1126-32
Manicklal, S., Emery, V. C., Lazzarotto, T., Boppana, S. B., and Gupta, R. K. (2013) The “silent” global burden of congenital cytomegalovirus. Clin Microbiol Rev, 26:86-102
Redwood, A. J., Messerle, M., Harvey, N. L., Hardy, C. M., Koszinowski, U. H., Lawson, M. A., and Shellam, G. R. (2005) Use of a murine cytomegalovirus K181-derived bacterial artificial chromosome as a vaccine vector for immunocontraception. J Virol, 79:2998-3008
Swanson, E. C., and Schleiss, M. R. (2013) Congenital Cytomegalovirus Infection: New Prospects for Prevention and Therapy: for Pediatric Clinics of North America: Advances in Evaluation, Diagnosis and Treatment of Pediatric Infectious Disease. Pediatr Clin North Am, 60:335-49
Wen, L., Y. Qiu, S. Cheng, X. Jiang, Y. P. Ma, W. Fang, W. Wang, J. Cui, Q. Ruan, F. Zhao, F. Hu, and M. H. Luo. (2018) Serologic and viral genome prevalence of HSV, EBV, and HCMV among healthy adults in Wuhan, China. J Med Virol, 90:571-81

What is claimed is:

1. A genetically engineered MCMV-K181 strain-derived tool virus for tracing virus infection, wherein the tool virus is a recombinant MCMV-K181 strain comprising:
a tracing elements expression cassette and a BAC backbone, wherein the BAC backbone and the tracing elements expression cassette are inserted together between M06 open reading frame and M07 open reading frame of MCMV-K181 strain genome;
wherein the tracing elements expression cassette comprises three tracing elements coding sequences; and wherein the tracing elements expression cassette from 5 ‘to 3’ sequentially comprises a first fluorescent protein, a first linker, a second fluorescent protein, a second linker, a luciferase and a polyA, and is transcribed into a transcript, resulting in three tracing elements after translation, namely, the first fluorescent protein, the second fluorescent protein and the luciferase.

2. The tool virus of claim 1, wherein the BAC backbone comprises a DNA replication origin ori, a repE gene, a sopA gene, a sopB gene, a sopC gene, an ori2 replicon, a prokaryotic transcription terminator, and an ampicillin resistance gene.

3. The tool virus of claim 1, wherein insertion site of the BAC backbone and tracing elements expression cassette between the M06 open reading frame and M07 open reading frame of MCMV-K181 strain genome is between 6522 and 6545.

4. The tool virus of claim 1, wherein the first and second fluorescent proteins are selected from the group consisting of GFP (green fluorescent protein), eGFP (enhanced green fluorescent protein), mGFP (membrane bound form of eGFP), sfGFP (super folder green fluorescent protein), mNeonGreen, StayGold, EYFP (enhanced yellow fluorescent protein), ECFP (enhanced cyan fluorescent protein) EBFP2 (enhanced blue fluorescent protein 2), tdTomato, MRFP (monomer red fluorescent protein), mRb3, mScarlt, DsRed, mCherry, Ypet, mKO, mkate and iRFP.

5. The tool virus of claim 4, wherein the first fluorescent protein is eGFP with an amino acid sequence represented by SEQ ID NO. 2, and the second fluorescent protein is ZsGreen1 with an amino acid sequence represented by SEQ ID NO. 4.

6. The tool virus of claim 1, wherein the luciferase is NanoLuc with an amino acid sequence represented by SEQ ID NO. 6.

7. The tool virus of claim 1, wherein the first linker and the second linker encode a linker polypeptide, wherein the linker polypeptide comprises at least adjacent glycine and proline.

8. The tool virus of claim 7, wherein the linker polypeptide is T2A with an amino acid sequence represented by SEQ ID NO. 8, or P2A with an amino acid sequence represented by SEQ ID NO. 10.

9. The tool virus of claim 1, wherein the polyA is SV40 pA with a nucleotide sequence represented by SEQ ID NO. 11.

10. The tool virus of claim 1, wherein the tracer factor expression cassette further comprises a promoter, which is selected from the group consisting of CMV promoter, EF1a promoter, SV40 promoter, PGK1 promoter, Ubc promoter, human beta actin promoter, CAG promoter and HIF alpha promoter.
